(19) United States
(12) Patent Application Publication
(10) Pub. No.: US 2020/0282042 A1
(43) Pub. Date: Sep. 10, 2020

(54) CLINICAL AND IMMUNOLOGIC BIOMARKERS FOR REGRESSION OF HIGH GRADE CERVICAL DYSPLASIA AND CLEARANCE OF HPV16 AND HPV18 INFECTION AFTER IMMUNOTHERAPY

(71) Applicant: INOVIO PHARMACEUTICALS, INC., Plymouth Meeting, PA (US)

(72) Inventors: Kimberly KRAYNYAK, Norristown, PA (US); Matthew MORROW, Bala Cynwyd, PA (US); Albert J. SYLVESTER, Croydon, PA (US)

(21) Appl. No.: 16/612,556

(22) PCT Filed: May 11, 2018
(86) PCT No.: PCT/US2018/032464

§ 371 Date: Nov. 11, 2019

Related U.S. Application Data
(60) Provisional application No. 62/504,858, filed on May 11, 2017.

(51) Int. Cl. A61K 39/12; C12N 15/86; G01N 33/50

(52) U.S. Cl. CPC A61K 39/12; A61K 2039/892; G01N 33/5088; C12N 15/86

(57) ABSTRACT

Methods for treating a subject infected with human papillomavirus and accompanying symptoms comprise assessing a sample obtained from the patient after the treatment is administered to detect the presence or absence of biomarkers associated with lesion regression and/or clearance of the virus from the subject.

### CROSS REFERENCE TO RELATED APPLICATIONS

This application is the National Stage Application of International Patent Application No. PCT/US2018/032464, filed May 11, 2018, which claims benefit of U.S. Provisional Application No. 62/504,858, filed May 11, 2017, the contents of which are incorporated here in their entirety.

### TECHNICAL FIELD

The present disclosure relates to methods of characterizing immune responses and identifying subjects who respond to immunotherapy.

### BACKGROUND

Disease caused by human papillomavirus (HPV) remains common despite preventive vaccines and screening strategies. Globally, persistent HPV infections cause one-third of infection-associated cancers. All squamous cancers of the cervix (SCC) are thought to arise from untreated intraepithelial disease, with particular emphasis on cervical intraepithelial neoplasia 2/3 (CIN2/3). While all squamous cancers of the cervix arise from untreated CIN2/3, not all CIN2/3 progress to cancer. Reported rates of spontaneous regression in a relatively short, prospective observational protocol prior to planned therapeutic resection 2/3, suggest that in some people endogenous immune responses can eliminate CIN2/3. Patients who do not exhibit spontaneous lesion regression are indicated for ablative or surgical treatment interventions including cryotherapy, cold-knife conization, or loop electrical excision procedure (LEEP). While effective, these interventions can have undesirable side effects including pain, cervical incompetence leading to preterm birth, and low infant birthweight. Moreover, these procedures may not clear infection by high-risk HPV (HR-HPV), thus leaving the patient at risk for recurrence of disease.

A Phase IIb double blind placebo controlled trial studied the effect of administering VGX-3100, a plasmid that encodes the E6 and E7 antigens of HPV16 and HPV18, on high grade dysplasia caused by infection with HPV16/18 (Trimble et al., Lancet, 386(10008): 2078-2088 (2015)). In that trial, VGX-3100 was administered via intramuscular immunization in the deltoid followed by in vivo electroporation using the CELLECTRA® device (Inovio, Plymouth Meeting, Pa.) at Weeks 0, 4 and 12 to patients with confirmed HPV16 and/or HPV18 infection and high grade cervical dysplasia (CIN2/3). VGX-3100 was efficacious as defined by the primary endpoint of CIN2/3 lesion regression and the secondary endpoint of lesion regression with concomitant elimination of HPV16/18 infection. It takes substantial time to observe lesion regression and HPV clearance in a subject, and earlier assessments would shorten the length of clinical trials and result in significant cost decreases and potentially shorten the time necessary to bring the therapeutic to market. There remains a need for methods for predicting the effectiveness of the therapy at much earlier time points. This application is directed to this and other important needs.

### SUMMARY

Method are provided herein for characterizing an immune response to an immunostimulatory composition comprising administering the immunostimulatory composition to a subject; obtaining a sample from the subject; and performing an assay on the sample to determine the presence of CD8+ cells, CD137+ cells, or both, wherein the cells express a detectable levels of perforin, granzyme A, granzyme B, or granulysin, or any combination thereof, wherein the presence of said cells indicate an immune response was initiated in response to the administered immunostimulatory composition.

Also provided herein are methods for identifying a subject having high-grade (CIN2/3) cervical dysplasia and HPV-16 and/or HPV-18 infection responsive to treatment with VGX-3100 comprising administering VGX-3100, obtaining at least one biological sample, and performing assays on the at least one biological sample to detect the presence of an intraepithelial lesion cytology and the absence of HPV-16 and/or HPV-18, wherein a combination of no intraepithelial lesion and no HPV-16 and/or HPV-18 are indicative of response.

Disclosed herein are methods for identifying a subject having high-grade (CIN2/3) cervical dysplasia and HPV-16 and/or HPV-18 infection responsive to treatment with VGX-3100 comprising administering VGX-3100, obtaining at least one biological sample, and performing an assay on the at least one biological sample to determine the presence of CD8+ cells, CD137+ cells, or both, wherein the cells express a detectable levels of perforin, granzyme A, granzyme B, or granulysin, or any combination thereof, wherein the presence of said cells indicate an immune response was initiated in response to the administered immunostimulatory composition.

### BRIEF DESCRIPTION OF THE DRAWINGS

FIG. 1A depicts a representative staining of CD8 in dysplastic cervical epithelium. FIG. 1B graphically depicts the number of CD8+ cells in dysplastic cervical tissue in VGX-3100 treated patients broken out by regression status, and FIG. 1C graphically depicts the number of CD8+ cells in dysplastic cervical tissue in VGX-3100 treated patients broken out by regression status concomitant with HPV clearance. Number of patients is listed under each group. For all figures, the number of patients assessed is listed under each group. Hatched boxes in pictures indicate area being more closely displayed within the inset. Arrows indicate positive staining. In all graphs, each dot represents one patient. The groups were compared with two-tailed Mann-Whitney tests for all analyses.

FIG. 2A depicts a representative staining of Foxp3 in immune infiltrates in dysplastic cervical epithelium. FIG. 2B graphically depicts the number of Foxp3+ cells in dysplastic cervical tissue in VGX-3100 treated patients broken out by regression status. FIG. 2C graphically depicts the number of Foxp3+ cells in dysplastic cervical tissue in VGX-3100 treated patients broken out by regression status concomitant with HPV clearance.

FIG. 3A graphically depicts the ratio of CD8+/Foxp3+ cells in dysplastic cervical tissue in VGX-3100 treated patients broken out by regression status. FIG. 3B graphically depicts the ratio of CD8+/Foxp3+ cells in dysplastic cervical tissue in VGX-3100 treated patients broken out by regression status concomitant with HPV clearance (right panel).

FIG. 4A depicts a representative staining of PD-L1 in immune infiltrates in dysplastic cervical epithelium. FIG. 4B graphically depicts the frequency of PD-L1 staining in dysplastic cervical tissue in VGX-3100 treated patients broken out by regression status FIG. 4C graphically depicts the frequency of PD-L1 staining in dysplastic cervical tissue in VGX-3100 treated patients broken out by regression status concomitant with HPV clearance.

FIG. 5A and FIG. 5B depict the output from flow cytometry analysis of CD137 from peripheral CD8+ T cells obtained from subjects that received a placebo and VGX-3100.

FIG. 6 depicts the output from flow cytometry analysis of granulysin, perforin, granzyme A and granzyme B from peripheral CD8+ T cells.

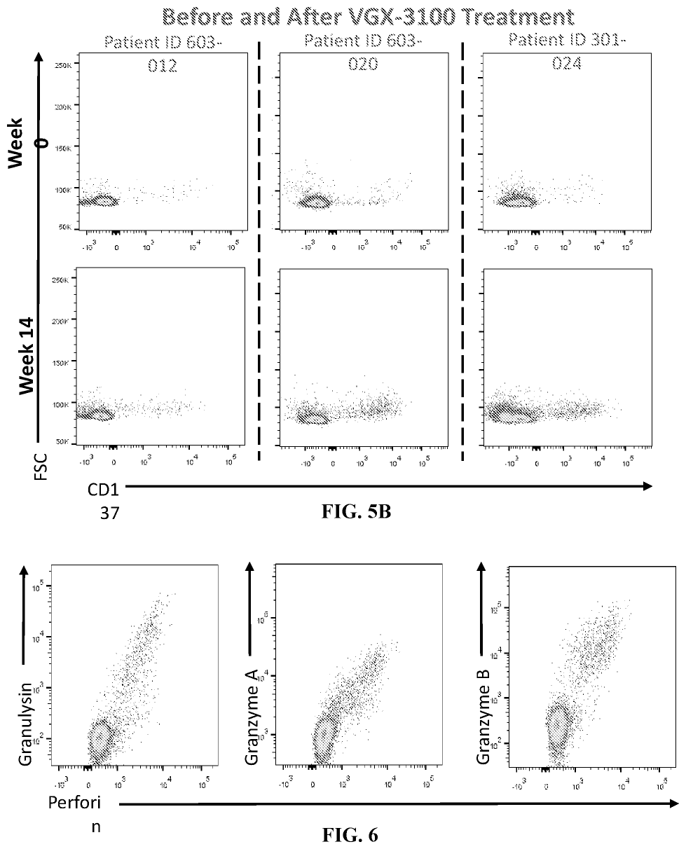

FIG. 7A graphically depicts the frequency of CD137 expression on VGX-3100 specific CD8+ T cells broken out by treatment allocation. FIG. 7B and FIG. 7C graphically depict the primary efficacy endpoint and secondary efficacy endpoint, respectively.

FIG. 8A and FIG. 8B graphically depict VGX-3100-induced specific expression of perforin within CD8+/CD137+ T cells broken out by primary efficacy endpoint or secondary efficacy endpoint, respectively. FIG. 8C and FIG. 8D graphically depict VGX-3100-induced specific expression of granulysin within CD8+/CD137+ T cells broken out by primary efficacy endpoint or secondary efficacy endpoint, respectively.

FIG. 9A and FIG. 9B graphically depict expression of perforin and granzyme A within CD8+/CD137+ T cells responding to HPV16 and HPV18 E6 and E7 broken out by primary efficacy endpoint or secondary efficacy endpoint, respectively. FIG. 9C and FIG. 9D graphically depict expression of perforin and granzyme B within CD8+/CD137+ T cells responding to HPV16 and HPV18 E6 and E7 broken out by primary efficacy endpoint or secondary efficacy endpoint, respectively.

FIG. 10A and FIG. 10B graphically depict expression of perforin within CD8+/CD137+ T cells responding to the E6 and E7 antigens only of the HPV type the patient is infected with broken out by primary efficacy endpoint or secondary efficacy endpoint, respectively. FIG. 10C and FIG. 10D graphically depict expression of granulysin within CD8+/CD137+ T cells responding to the E6 and E7 antigens only of the HPV type the patient is infected with broken out by primary efficacy endpoint or secondary efficacy endpoint, respectively.

FIG. 11A and FIG. 11B graphically depict expression of perforin and granzyme A within CD8+/CD137+ T cells responding to the E6 and E7 antigens only of the HPV type the patient is infected with broken out by primary efficacy endpoint or secondary efficacy endpoint, respectively. FIG. 11C and FIG. 11D graphically depict expression of perforin and granzyme B within CD8+/CD137+ T cells responding to the E6 and E7 antigens only of the HPV type the patient is infected with broken out by primary efficacy endpoint or secondary efficacy endpoint, respectively. For all graphs, all visible symbols represent a single patient, with other patients being represented within the box and whiskers (where relevant). The changes in results between study weeks were compared with two-tailed Wilcoxon signed-rank tests for all comparisons.

FIG. 12. Results from In Situ Hybridization and PCR Assays for HPV Type. (A) Left: H&E staining identifying the presence of cervical dysplasia. Right: representative positive staining patterns for presence of HPV16 or HPV18 in cervical lesions present at Week 36. Black arrows denote positive signal.

FIG. 13A is a pie chart showing 13 patients treated with VGX-3100 who were negative for HPV16 or HPV18 by at least one assay (ISH or PCR) at Week 36. FIG. 13B presents tabulated data for six patients were negative in both assays. PCR confirmed the presence of other HR-HPV types present in four of six patients.

FIGS. 14A to 19B depicts immunohistochemical analysis of immune infiltration and immunosuppressive factors in cervical tissue at Week 36. FIG. 14A depicts CD137 staining on immune infiltrates in cervical epithelium at Week 36. FIG. 14B graphically depicts the number of CD137+ cells in cervical epithelial tissue in untreated and VGX-3100 treated patients broken out by regression status. FIG. 14C graphically depicts the number of CD137+ cells in cervical epithelial tissue in untreated and VGX-3100 treated patients broken out by regression status concomitant with HPV clearance. Hatched boxes indicate area being more closely displayed within the inset.

FIG. 15A depicts CD103 staining on immune infiltrates in cervical epithelium at Week 36. FIG. 15B graphically depicts the number of CD103+ cells in cervical epithelial tissue in untreated and VGX-3100 treated patients broken out by regression status. FIG. 15C graphically depicts the number of CD103+ cells in cervical epithelial tissue in untreated and VGX-3100 treated patients broken out by regression status concomitant with HPV clearance. Hatched boxes indicate area being more closely displayed within the inset. ‡ p=0.055

FIG. 16A graphically depicts the increases in Foxp3 infiltration from Week 0 to Week 36 in CIN2/3 epithelial and stromal tissue in untreated and VGX-3100 treated patients broken out by regression status. FIG. 16B graphically depicts the increases in Foxp3 infiltration from Week 0 to Week 36 in CIN2/3 epithelial and stromal tissue in untreated and VGX-3100 treated patients broken out by regression status concomitant with HPV clearance. FIG. 16C graphically depicts the CD8/+Foxp3+ ratios in cervical epithelium at Week 36 in cervical epithelial tissue in untreated and VGX-3100 treated patients broken out by regression status. FIG. 16D graphically depicts CD8/+Foxp3+ ratios in cervical epithelium at Week 36 in untreated and VGX-3100 treated patients broken out by regression status concomitant with HPV clearance.

FIG. 17A graphically depicts the increases in PD-L1 expression from Week 0 to Week 36 in CIN2/3 epithelial and stromal tissue in untreated and VGX-3100 treated patients broken out by regression status. FIG. 17B graphically depicts the increases in PD-L1 expression from Week 0 to Week 36 in CIN2/3 epithelial and stromal tissue in untreated and VGX-3100 treated patients broken out by regression status concomitant with HPV clearance.

FIG. 18A depicts perforin staining on immune infiltrates in cervical epithelium at Week 36. FIG. 18B graphically depicts the increases in perforin infiltration from Week 0 to Week 36 in cervical epithelial tissue in untreated and VGX-3100 treated patients broken out by regression status. FIG. 18C graphically depicts the increases in perforin infiltration from Week 0 to Week 36 in cervical epithelial tissue in untreated and VGX-3100 treated patients broken out by regression status concomitant with HPV clearance. Hatched boxes in tissue photographs indicate area being more closely displayed within the inset. Arrows indicate positive staining. The changes in results between study weeks were compared with two-tailed Wilcoxon signed-rank tests for comparisons in which statistical analysis was performed.

FIGS. 19A and 19B graphically depict immunohistochemical analysis of CD8 infiltration at study start. FIG. 19A shows the frequency of CD8 positive cells/mm2 as broken out by achievement of the primary endpoint of histopathological regression of CIN2/3 to CIN1 or WNL. FIG. 19B shows the frequency of CD8 positive cells/mm2 as broken out by achievement of the primary endpoint of histopathological regression of CIN2/3 to CIN1 or WNL concomitant with elimination of HPV16/18 infection.

FIG. 20 presents a table breakdown of HPV16 and HPV18 positivity at enrollment of the study. Patients are represented as being HPV16 positive only (HPV16+ and HPV18−), HPV18 positive only (HPV16− and HPV18+) or positive for both viruses (HPV16+ and HPV18+). The bottom row represents the total patients in the study. Columns are broken into placebo and VGX-3100 cohorts and total patients in the study.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

When a range of values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods, which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include the plural.

The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

The term “subject” as used herein refers to any animal, but in particular humans. Thus, the methods are applicable to human and nonhuman animals, although preferably used most preferably with humans. “Subject” and “patient” are used interchangeably herein.

As used herein, “treating” and like terms refer to reducing the severity and/or frequency of HPV infection symptoms, for example and especially lesions; eliminating HPV infection symptoms, especially lesions; and/or clearing HPV virus from the subject.

Clinical trials are implemented to discern the efficacy and safety of therapeutics by studying the effects of the therapeutics in a large population. These studies are costly and time consuming endeavors. Methods that reduce the time necessary to assess therapeutics would reduce these costs and potentially hasten the availability of the therapies to the general product. Provided herein are methods for determining if an immunotherapeutic elicits the recruitment of immune system cells having particular expression profiles. It is disclosed herein that these profiles, if detected after administration of the therapeutic, are accurate predictors of clinical outcome. In some aspects, the profiles can be detected at 10, 12, 14, or 16 weeks after first administration of the therapeutic. In other aspects, the immune cells exhibiting the desired profiles can be detected as early as 8, 6, or even 4 weeks.

Provided herein are methods of characterizing an immune response to an immunostimulatory composition comprising administering the immunostimulatory composition to a subject, obtaining a sample from the subject; and performing an assay on the sample to determine the presence of CD8+ cells, CD137+ cells, or both, wherein the cells express a detectable levels of perforin, granzyme A, granzyme B, or granulysin, or any combination thereof, wherein the presence of said cells indicate an immune response was initiated in response to the administered immunostimulatory composition. In some aspects of the present disclosure, the sample is a peripheral blood sample. In other aspects, the sample is a cytological sample. Because certain diseases caused by HPV infection manifest with aberrant cervix cell regulation or morphology of cervix cells, in some aspects, the cytological sample is a cervix sample.

In some embodiments of the present disclosure, the methods for characterizing an immune response further comprise testing the sample for the presence of programmed death ligand 1 (PD-L1), which is suspected of suppressing immune system function. In some aspects, detection of PD-L1 can warrant a prediction that the immune response generated by the therapy will be insufficient due to the immunosuppressive effects of this protein.

In some embodiments of the present disclosure, the immunotherapy comprises administering a DNA plasmid encoding at least one antigen of HPV-16, HPV-18, or both. In some aspects of the embodiment, the DNA plasmid encodes an E6 and an E7 antigen of HPV-16, an E6 and an E7 antigen of HPV-18, or both. In some aspects, the immunotherapy is VGX-3100.

Another embodiment of the present disclosure provides methods for identifying a subject having high-grade (CIN2/3) cervical dysplasia and HPV-16 and/or HPV-18 infection responsive to treatment with VGX-3100 comprising administering VGX-3100, obtaining at least one biological sample, and performing assays on the at least one biological sample to detect the presence of an intraepithelial lesion cytology and the absence of HPV-16 and/or HPV-18, wherein a combination of no intraepithelial lesion and no HPV-16 and/or HPV-18 are indicative of response. In some aspects of the embodiment, the sample is a peripheral blood sample, while in other aspects the sample is a cytological sample. In addition, in some aspects, the cytological sample is a cervix sample.

Methods are also provided for identifying a subject having high-grade (CIN2/3) cervical dysplasia and HPV-16 and/or HPV-18 infection responsive to treatment with VGX-3100 comprising administering VGX-3100, obtaining at least one biological sample, and performing an assay on the at least one biological sample to determine the presence of CD8+ cells, CD137+ cells, or both, wherein the cells express a detectable levels of perforin, granzyme A, granzyme B, or granulysin, or any combination thereof, wherein the presence of said cells indicate an immune response was initiated in response to the administered immunostimulatory composition. In some aspects of the embodiment, the sample is a peripheral blood sample, while in other aspects the sample is a cytological sample. In addition, in some aspects, the cytological sample is a cervix sample.

In some embodiments of the present disclosure, the immunotherapy comprises administering a DNA plasmid encoding at least one antigen of HPV-16, HPV-18, or both. In some embodiments, the DNA plasmid encodes an E6 and an E7 antigen of HPV-16, an E6 and an E7 antigen of HPV-18, or both.

### EXAMPLES

The following examples are provided to further describe some of the embodiments disclosed herein. The examples are intended to illustrate, not to limit, the disclosed embodiments.

### Example 1: Pre-Existing Immune Cell Infiltration in Cervical Lesions at Study Entry do not Associate with Histologic Regression

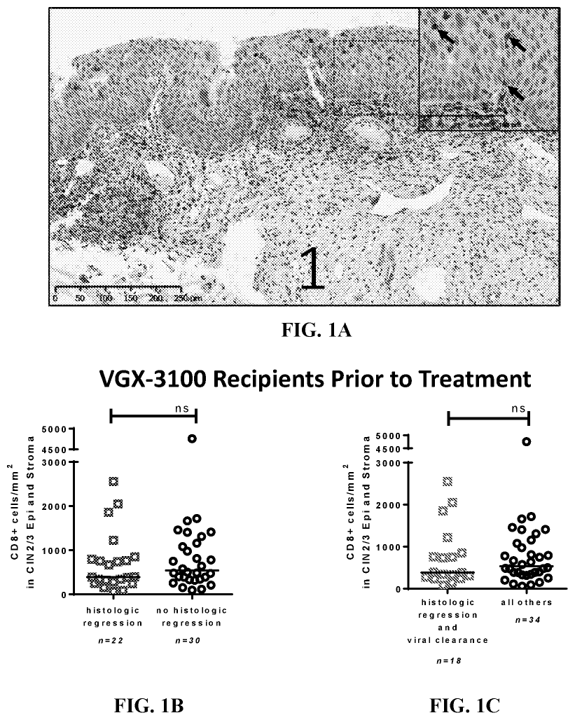

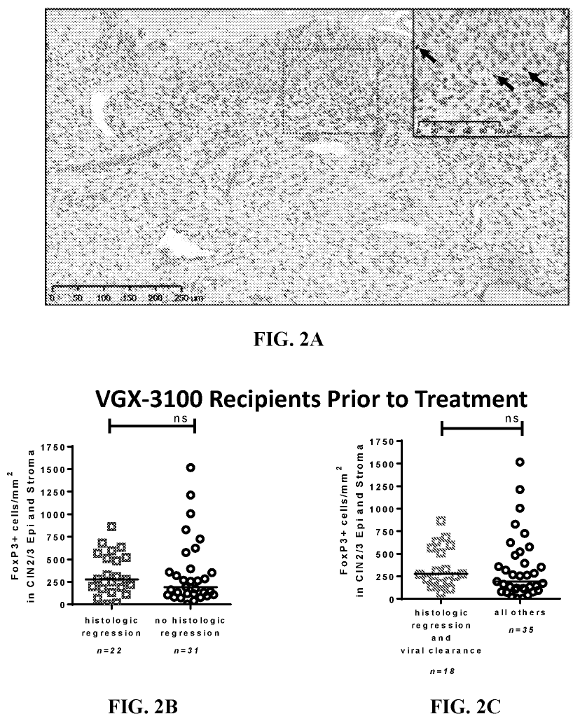

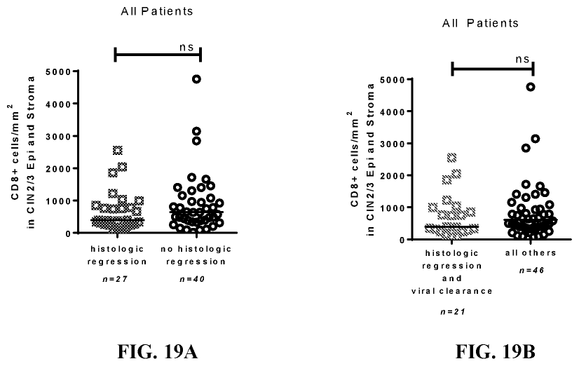

Treatment with VGX-3100 has been previously reported to result in histologic regression of high grade cervical dysplasia (CIN2/3) and clearance of HPV16 and/or HPV18 in a Phase IIb double blind, placebo-controlled trial (Trimble et al., Lancet, 386(10008): 2078-2088 (2015)). In this current study, cervical tissue obtained from these patients prior to dosing with VGX-3100 was analyzed to determine if any pre-existing factors were associated with the subsequent success or failure in treating CIN2/3. In patients with CIN2/3 observed over a 15-week window prior to planned resection, the intensity of intraepithelial CD8+ T cell infiltrates in lesional mucosa has been previously reported to be associated with the likelihood of subsequent histologic regression. In the current study, in patients treated with VGX-3100, no statistical association was found between the intensity of pre-treatment CD8+ immune infiltrates and subsequent treatment success in lesional epithelium and stroma (FIGS. 1B and 1C respectively). Indeed, study entry CD8+ tissue infiltrates did not associate with lesion regression in either VGX-3100 or placebo recipients (FIGS. 19A and 19B). Biopsies from study entry were assessed for the presence of immunosuppressive factors that might blunt immune responses generated by VGX-3100 and thus possibly influence treatment outcomes. Immunohistochemical (IHC) staining was performed for the Foxp3 transcription factor indicative of regulatory T cells (Treg) (FIG. 2A).

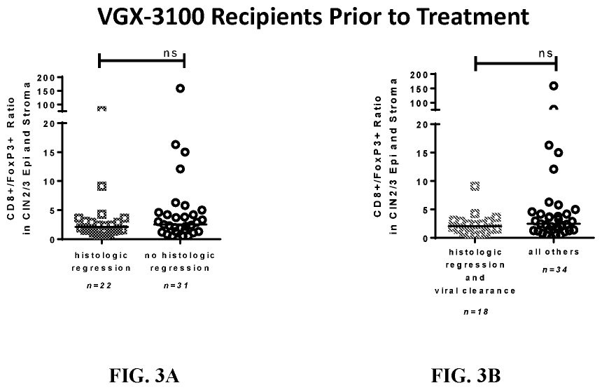

Prior to treatment, the highest frequency of cells that were Foxp3+ in dysplastic tissue were found in treated patients who did not achieve the primary or secondary endpoint compared to those who did, although there was no significant difference between the groups (FIGS. 2B and 2C, respectively). As the ratio of CD8 to Foxp3 has been shown to change in response to immunotherapy and may be informative for clinical prognosis in some diseases (Semeraro et al., Oncoimmunology, 5(10): e1218106 (2016); Zhu et al., Oncotarget, 7(44):71455-65 (2016), this analytical approach was also used in the present study. The CD8+:Foxp3+ ratio at study entry did not associate with either the primary or secondary endpoints (FIGS. 3A and 3B).

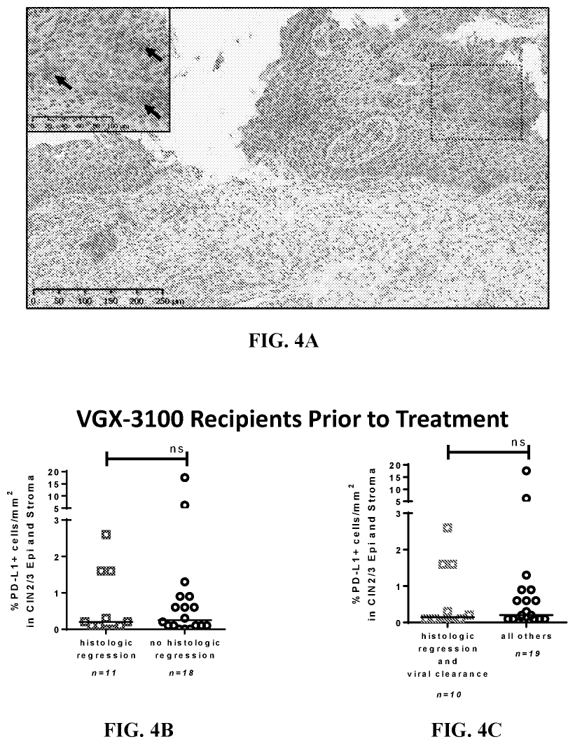

The immunosuppressive ligand PD-L1 is expressed by a variety of cell types including dysplastic, neoplastic, and immune cells in various disease states including cervical dysplasia. The pattern of PD-L1 staining in dysplastic tissue at study entry was restricted to dysplastic epithelium, and was predominantly cytoplasmic (FIG. 4A), consistent with other reports of PD-L1 expression profiles in cervical dysplasia. The highest frequency of cells that were PD-L1+ in dysplastic tissue were found in treated patients who did not achieve the primary or secondary endpoint compared to those who did, although there was no significant difference between the groups (FIGS. 4B and 4C, respectively). Neither the intensity of CD8+ or Treg cellular infiltration, nor expression of PD-L1 in pre-treatment cervical lesions were either positively or negatively associated with the eventual success or failure of VGX-3100 to treat high grade cervical dysplasia.

### Example 2: Real-Time Examination of Cytology and Virology are Clinical Predictors of Treatment Outcome

In addition to analyzing pre-existing factors that might influence treatment outcomes, longitudinal cytologic and/or virologic testing performed during the study were explored as possible predictors of ultimate treatment outcomes. Specifically, the time period following the completion of VGX-3100 dosing but prior to the efficacy assessment was explored. The utility of cytology and HPV typing data collected from cervical swabs for use as predictors of treatment success or failure was also assessed. The diagnosis of “No intraepithelial lesion” at Week 14 was compared with any abnormal diagnosis, at that time point, including “high grade squamous intraepithelial lesion (HGSIL)”, “atypical squamous cells, cannot rule out HGSIL (ASC-H)” and performed sensitivity, specificity, and positive and negative predictive value calculations. Normal cytology (NIL) alone was not a strong predictor of ultimate histologic outcome, only 32% of patients with this cytology ultimately had histologic regression by Week 36 (Table 1). However, in patients with high grade cytology (HSIL/ASC-H) at Week 14, only 17% would ultimately had histologic regression by Week 36.

The predictive value of detecting HPV16 and/or 18 at the same time point was also assessed alone or in combination with the cytology data. Patients whose HPV16/18 had become undetectable at Week 14 were likely to regress by Week 36; 84% of these patients had histologic regression. While the sensitivity and specificity of these individual measures were not uniformly high, the combination of Week 14 cytology and virology was a much stronger predictor of ultimate histologic regression results. The finding of normal (NIL) cytology and HPV16/18 clearance at Week 14 was much more likely to predict histologic regression at Week 36 than a finding of HGSIL/ASC-H and persistence of HPV16/18 (negative predictive value 94%; sensitivity 96%). Taken together, these data suggest that the combination of clinical cytological and virological assessments from cervical swabs at Week 14 have high predictive value for lesion regression status at Week 36. Table 1 presents the results of these analyses from cytology and virology performed at Week 14, which is 2 weeks following the final dose of VGX-3100.

TABLE 1
Week 14 Cytological (Pap Smear and Virological (Roche Linear Array) Results

| | | | Negative | Positive | | |
|---|---|---|---|---|---|---|
| | Non- | | Predictive | Predictive | | |
| | Regressors | Regressors | Value | Value | Sensitivity | Specificity |
| NIL | 10 | 21 | 68% | 63% | 81% | 45% |
| Not NIL | 44 | 26 | | | | |
| HGSIL/ASC-H | 29 | 6 | 62% | 83% | 54% | 87% |
| Not | 25 | 41 | | | | |
| HGSIL/ASC-H | | | | | | |
| HPV 16/18 | 5 | 27 | 84% | 68% | 91% | 54% |
| Clearance | | | | | | |
| No HPV 16/18 | 49 | 23 | | | | |
| Clearance | | | | | | |
| HGSIL/ASC-H | 26 | 6 | 94% | 81% | 96% | 73% |
| and No | | | | | | |
| HPV16/18 | | | | | | |
| Clearance | | | | | | |
| NIL and | 1 | 16 | | | | |
| HPV16/18 | | | | | | |
| Clearance | | | | | | |

NIL—No Intraepithelial Lesion
HGSIL—High Grade squamous Intraepithelial Lesion
ASC-H—Atypical Squamous Cells, cannot rule out High Grade
Negative Predictive Value is the probability of regression given a negative Week 14 result, which are the following: NIL, Not HGSIL/ASCH, HPV 16/18 Clearance, and NIL and HPV 16/18 Clearance
Positive Predictive Value is the probability of non-regression given a positive Week 14 result, which are the following: Not NIL, HGSIL/ASCH, No HPV 16/18 Clearance, and HGSIL/ASCH and No HPV 16/18 Clearance

### Example 3: Genotype-Specific Peripheral Blood CD8+ T Cell Responses after the Third Dose of VGX-3100 Predict Treatment Success

While cytological and virological assessments had strong negative and positive predictive values for lesion regression status, the strength of these assessments applied to approximately half of the patients in the trial receiving VGX-3100 (49 of 101 evaluable patients). To identify additional factors that were predictive of treatment success, peripheral blood immune responses to VGX-3100 antigens at Week 14 (two weeks following the third and final dose of VGX-3100) was assessed. Post-immunization signatures at Week 14 were also analyzed for their ability to predict histologic regression status prior to the definitive determination at Week 36.

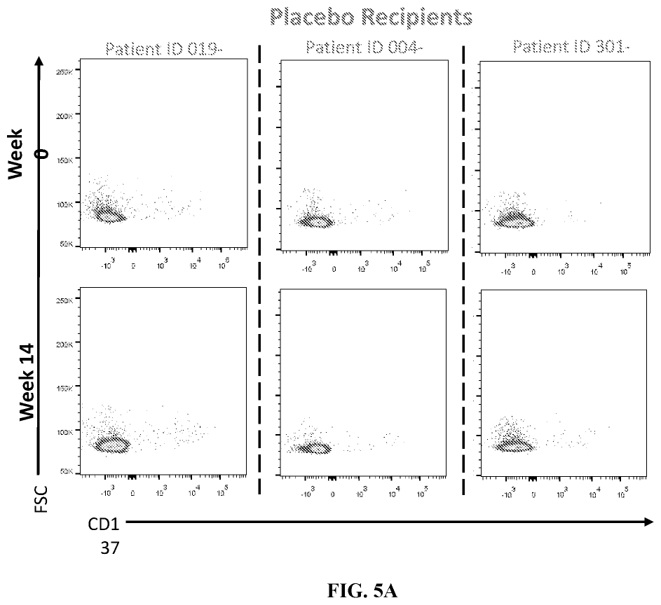

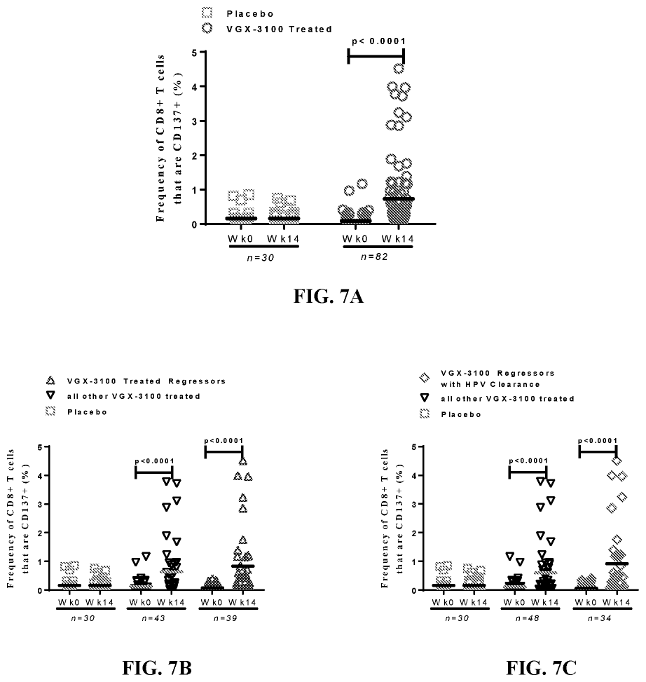

Using multiparametric flow cytometry, to determine if quantification of the frequency of CD8+ T cells that were specific for VGX-3100 would be sufficient to discriminate between patients who met primary or secondary endpoints and those who did not, CD137 staining was employed. CD137 is a marker used to identify CD8+ T cells activated by their cognate antigen (FIGS. 5A and 5B). While treatment with VGX-3100 significantly increased the frequency of HPV16/18 specific CD8+ T cells (FIG. 7A top panel), there was no significant difference in the magnitude of response at this time point, between treated patients who would go on to meet the primary or secondary endpoints from those who did not. In contrast, a significant post-treatment increase in the frequency of CD8+/CD137+ cells in patients treated with VGX-3100, irrespective of histologic regression outcome was observed (FIGS. 7B and 7C).

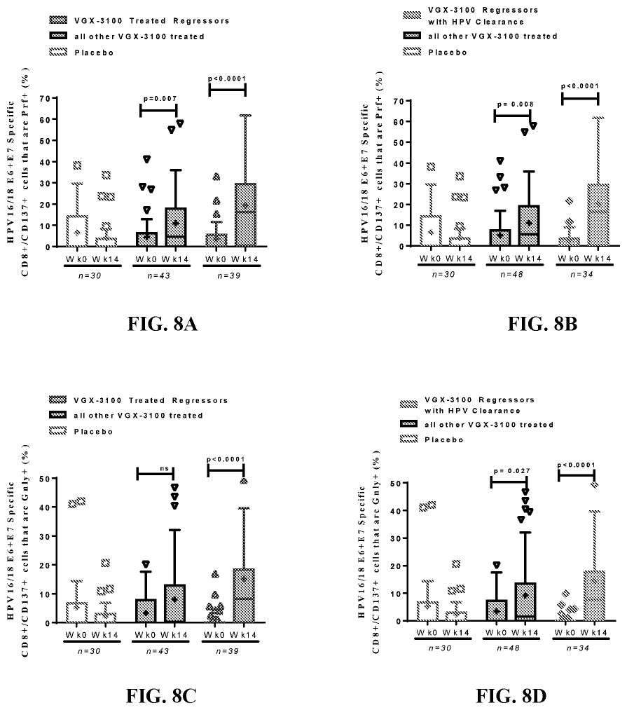

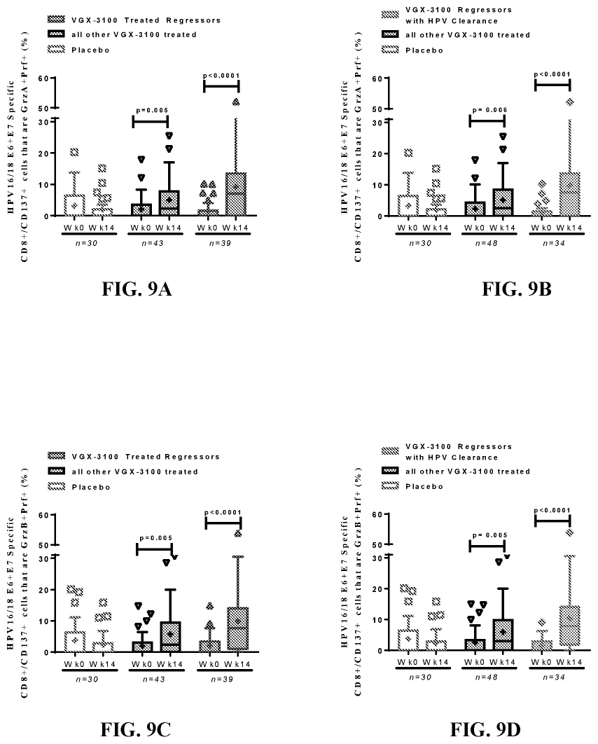

Because further characterizing the functionality of these CD8+ T cells might aid in identifying an immune phenotype that discriminates between treated patients whose lesions do or do not regress or clear HPV, antigen-activated CD8+ T cells (CD137+) were assessed for markers associated with lytic function: granzyme A, granzyme B, perforin and granulysin (FIGS. 5A and 5B). Expression of these lytic markers in the CD8+CD137+ population significantly increased in both treated patients who met primary and secondary endpoints as well as in treated patients who did not, in all but one analysis (analysis of granulysin for regression endpoint), suggesting that antigen-activated CD8+ T cell lytic potential did not correspond to lesion regression (FIGS. 8A to 9D).

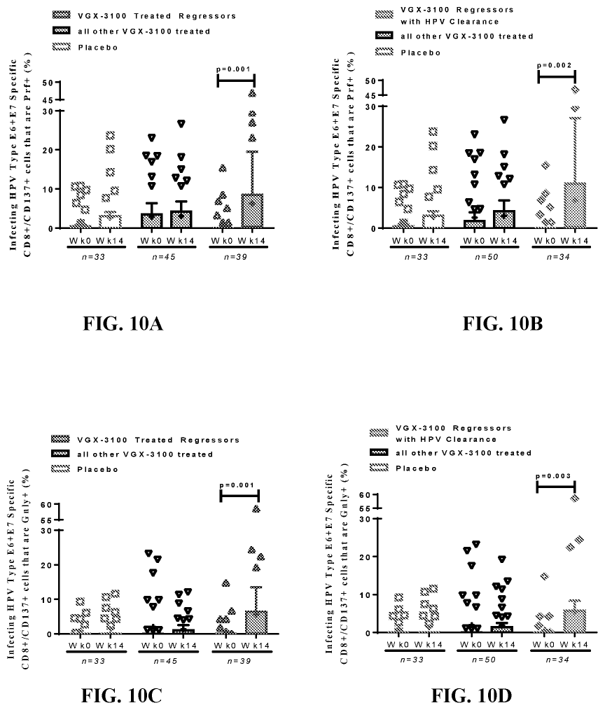

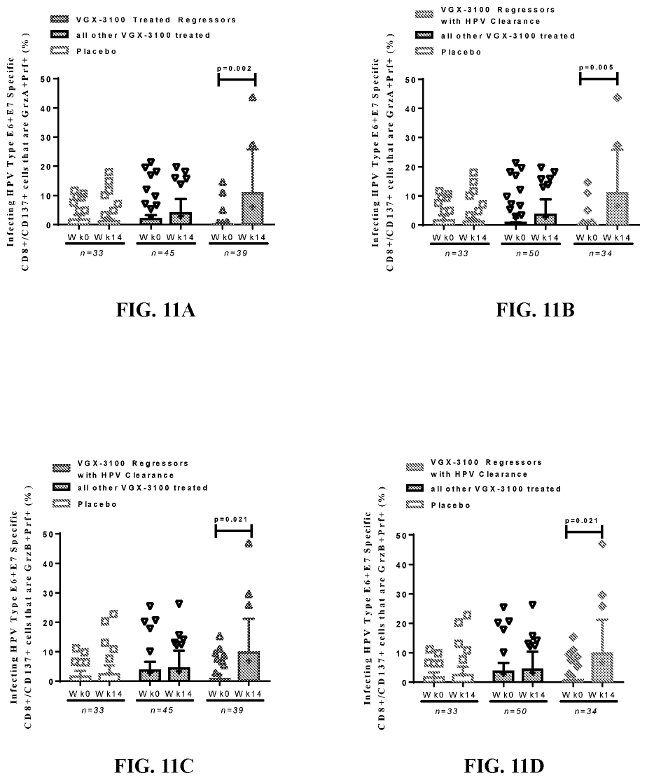

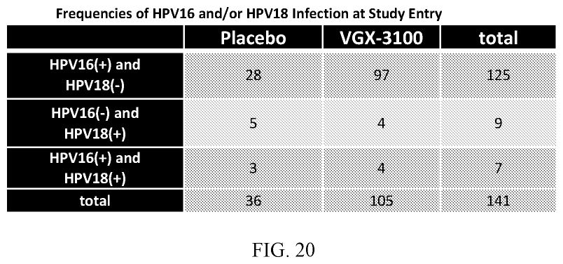

VGX-3100 targets four HPV antigens: E6 and E7 from HPV16 and E6 and E7 from HPV18. The majority of patients included in the efficacy analyses were infected with HPV16 alone (89%), while a minority were infected with either HPV18 alone (6%), or co-infected with both HPV genotypes (5%) (FIG. 20). Measured immune responses directed against some antigens may have confounded the analysis because they were being weighed against clinical benefit without being relevant to the infecting HR-HPV type of an individual patient. Therefore, to perform the most focused and relevant evaluation, additional analyses were performed which excluded immune responses against HPV16 antigens or HPV18 antigens if the patient did not have an active infection with that HR-HPV type. Analyses focused on immune responses relevant only to the HR-HPV type causing the high grade dysplasia in each patient showed that the frequency of lytic protein expression within the CD8+CD137+ T cell population that was specific for the HR-HPV the patient was infected with were significantly increased only in treated patients who met primary or secondary endpoints. This observation suggested that lytic potential in peripheral CD8+ T cells specific to the infecting HPV type is predictive of treatment success with VGX-3100 (FIGS. 10A to 11D). The discriminatory potential of this analysis was noted for perforin and granulysin expression as well as co-expression of granzyme A and perforin, and co-expression of granzyme B with perforin (FIGS. 10A to 11D).

### Example 4: Contribution of HPV Genotypes Other than HPV16 and HPV18 to Treatment Failures: Retrospective Analysis

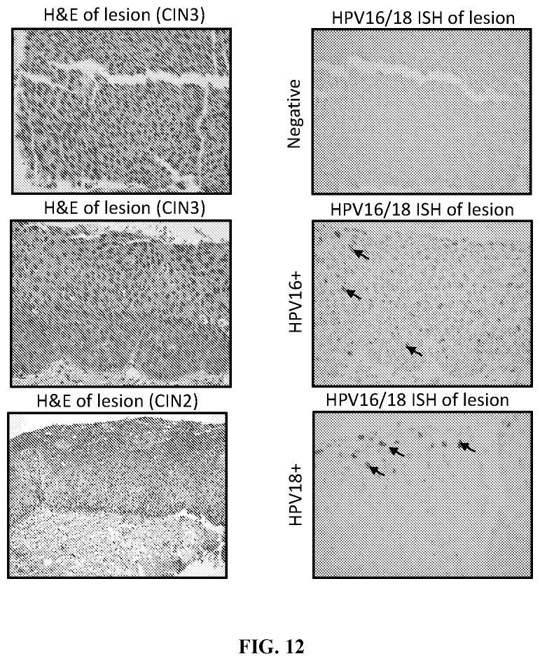

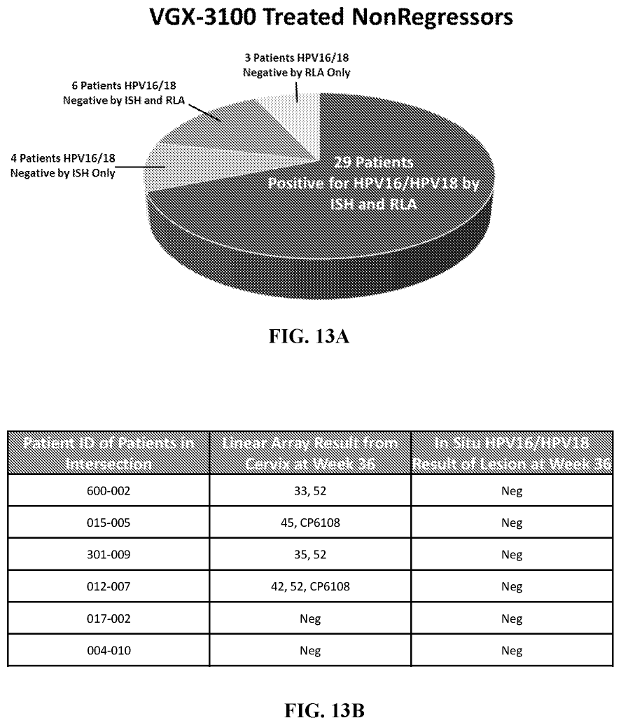

After establishing that HPV type specificity was a key component of a peripheral correlate of successful treatment with VGX-3100, direct in situ HPV typing was performed on dysplastic lesions that remained present in treated patients at the time of the efficacy assessment (Week 36) to determine if unresolved lesions were HPV16 or HPV18 positive (FIG. 12). In this trial, 56% of patients had mixed infections at study entry. However, assessment of HPV was performed on exfoliated cell samples. Because VGX-3100 was designed to treat high grade dysplasia driven by HPV16 or HPV18 but not other HR-HPV types, persistent lesions that were not positive for HPV16 or HPV18 might be due to non HPV16/18 types that were also present at diagnosis and may account for a portion of treatment failures noted in the trial. 42 of the 54 patients treated with VGX-3100 whose lesions had not regressed had sample evaluable for in situ analysis. Of these 42 patients, approximately one quarter (10) were found to have persistent high grade lesions that were not HPV16 or HPV18 positive by in situ hybridization (FIG. 13A), and 6 of those patients were also negative by PCR from a cervical swab (FIG. 13B). Further analysis of the PCR data of the cervical swab samples revealed that four of these patients were positive for other HR-HPV types (FIG. 13B). Agreement of these two independent assays and the presence of other HR-HPV types by PCR suggests that there are a subset of patients who were formally classified as VGX-3100 treated non-regressors, but may not have been true treatment failures since HPV16/18 was undetectable, suggesting that in these patients, persistent dysplasia was likely due to infection with another high risk HPV type. However, this observation accounts for only 11% of treatment failures in the VGX-3100 treated cohort, and thus other analyses of samples taken after the efficacy assessment were performed.

### Example 5: Retrospective Assessment of Tissue Infiltrating Immune Cell Subsets Predict Treatment Outcome

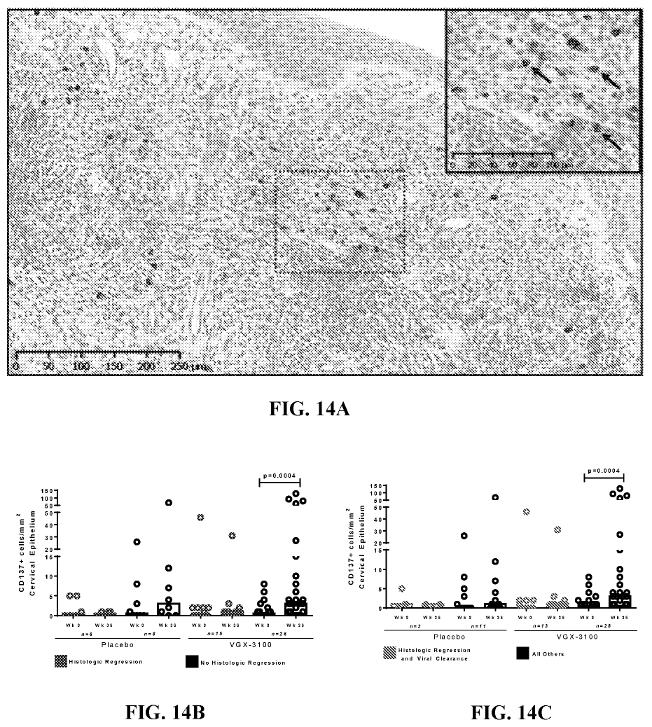

The analysis of cervical tissue demonstrated that while pre-treatment CD8+ infiltrates were not predictive of histologic response (FIGS. 1A to 1C), previous reports demonstrated statistically significant increases in CD8+ immune infiltration into normal cervical tissue in VGX-3100-treated patients whose lesions regressed histologically and also cleared virus (Trimble et al.). The quality of this response was further assessed by performing quantitative digital image post-hoc analyses of markers associated with antigen-induced activation, and differentiation into tissue-resident memory T cells. Cervical epithelial tissue obtained prior to treatment with VGX-3100 and obtained at the Week 36 efficacy assessment was tested for the presence of cells expressing CD137. Virological and cytological data (Table 1) suggest that the majority of treatment successes may have exhibited lesion regression and viral clearance as early as Week 14 and thus evidence of an active immune response might be difficult to detect in cervical tissue at Week 36. While it was hypothesized that some residual activated cells might still be detected within the cervical epithelium, no significant elevation in CD137+ infiltrates from Week 0 to Week 36 was observed in patients with either lesion alone or concurrent lesion regression and viral clearance (FIGS. 14A and 14B). This observation suggested that enough time had indeed passed after resolution of infection and associated pathology for an active immune response to wane and tissue homeostasis to be reestablished. However, VGX-3100 treated patients with persistent lesions had significant increases in the intensity of CD137+ infiltrates in residual dysplastic cervical tissue at Week 36 (FIGS. 14A to 14C), while patients in the placebo cohort whose lesions persisted to Week 36 did not. This observation suggests that VGX-3100 induced an immune response characterized by CD137 expression in dysplastic epithelium that was still detectable in lesions that had not yet regressed by the Week 36 endpoint evaluation.

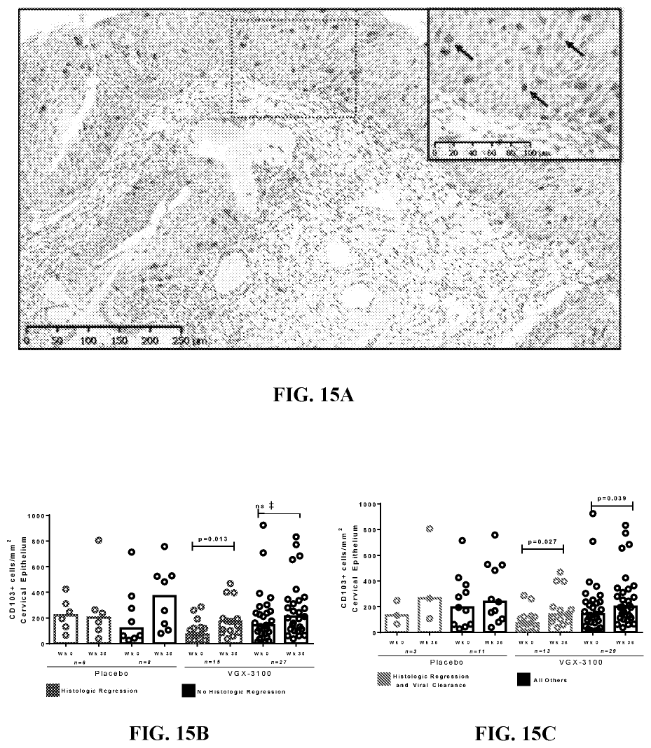

The increase in CD137 expression in cervical tissue from VGX-3100 treated patients suggests that an active immune response was once present in lesions that regressed with elimination of HPV16/18 infection, but that this response had waned by Week 36 owing to a complete resolution of disease prior to sampling. If this were the case, one would hypothesize that an increase in tissue resident memory T cells (Trm) that had converted from an activated effector state to a memory state would still be observable in the cervical epithelia of these patients. As CD103 expression is a hallmark of intraepithelial CD8+ Trm cells, the intensity of CD103 infiltration in cervical epithelium taken prior to VGX-3100 dosing and at Week 36 was compared. VGX-3100 treated patients whose lesions regressed and HPV16/18 was cleared showed significant increases in the frequency of intraepithelial CD103+ cells in residual normal mucosa (FIGS. 15A to 15C). While the frequency of CD103+ cells was also elevated in treated patients whose lesions did not regress, the magnitude of these increases did not reach significance (p=0.055). Patients who had histologic regression, but failed to achieve the secondary endpoint of concomitant viral clearance also had statistically significant elevation of mucosal CD103+ infiltrates (p=0.039). Patients who received placebo did not have significant increases in CD103+ tissue infiltrates (FIGS. 15A to 15C). These data suggest that the active immune response in treated patients whose lesions regressed had converted to a resident memory phenotype by Week 36 and that the ongoing immune response noted in the treated non-regressors may also have been converting to a memory response as well.

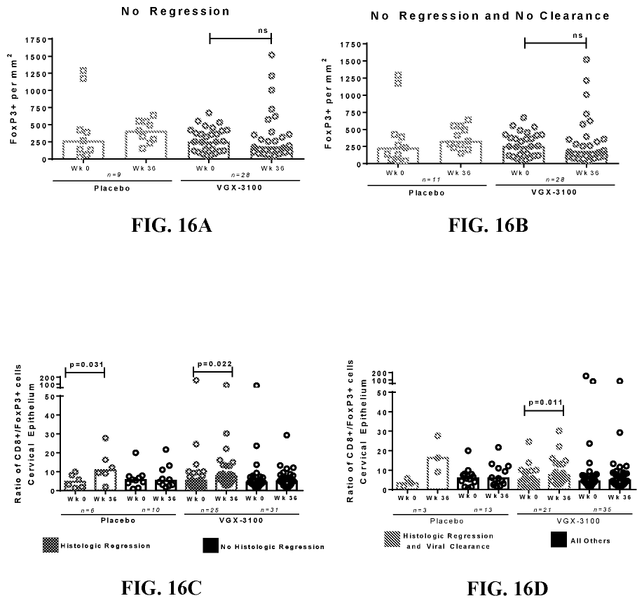

The increase in CD137+ and CD103+ immune infiltration observed in the cervical tissue of VGX-3100 treated non-regressors was paradoxical, because one might predict that immune infiltration of this nature would be a hallmark of patients who would be likely to regress their lesions, not a hallmark of lesion persistence. If an immune response was present in the cervical epithelium of patients with persistent disease, there could be an underlying immunoregulatory or immunosuppressive mechanism at work preventing efficient effector function. The assessment of Treg and PD-L1 expression at baseline had indicated that neither were predictive of treatment failure (FIGS. 1A to 4C). However, it has been reported that an infiltrating effector immune response can result in increases in either Treg infiltrates or PD-L1 expression. Thus Treg infiltration and PD-L1 expression in dysplastic lesions before and after VGX-3100 treatment (Week 36 timepoint) was compared. In the residual lesions of VGX-3100 treated non-regressors, Foxp3+ infiltrates did not increase significantly between Week 0 and Week 36. This observation suggested that VGX-3100 did not elicit mucosal Foxp3+ infiltrates (FIGS. 16A to 16D). Indeed, because of increases in CD8+ infiltrates, the CD8:Foxp3 ratio increased in VGX-3100-treated regressors from Week 0 to Week 36 (FIGS. 16A to 16D).

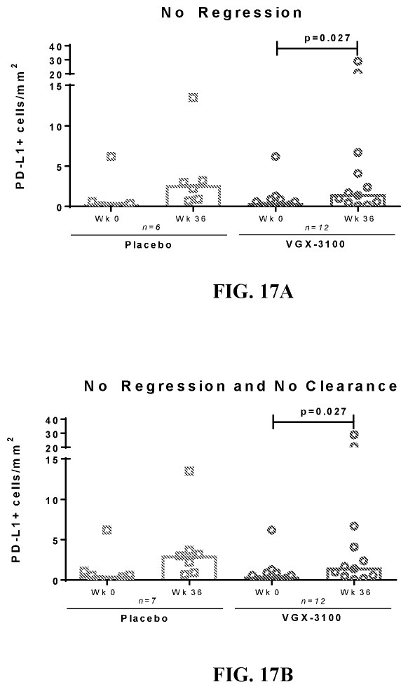

In contrast, in VGX-3100-treated non-regressors, PD-L1 expression in lesional tissue was significantly increased from Week 0 to Week 36 (FIGS. 17A and 17B). PD-L1 expression may be induced by a variety cytokines, including TNFα and IFN. Thus, the increase in PD-L1 expression in residual lesions suggested the ongoing presence of an active infiltrating effector immune response. In patients whose lesions did regress, PD-L1 expression did not change significantly (data not shown). These data suggest that upregulation of PD-L1 in lesional tissue of VGX-3100 treated patients may have played a role in treatment failure, although the specific impact of this upregulation is speculative as most evaluable patients displayed less than 5% staining of this immunosuppressive ligand by the time tissue was collected at Week 36.

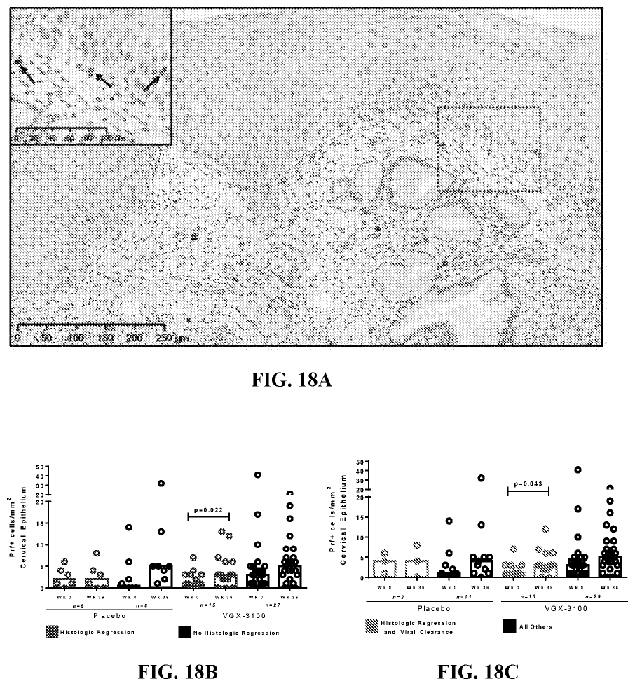

Lastly, the cervical tissue for markers that were associated with treatment success were evaluated. As the peripheral immune analysis had revealed that changes in expression of granulysin or perforin were the strongest predictors of concomitant lesion regression and clearance of HPV16 or HPV18, these cytolytic effector molecules were targeted for further analysis in cervical tissue. Confirmation of the presence of a similar signature in cervical tissue could possibly confirm that the responses noted in the periphery were mechanistically relevant for VGX-3100 driven efficacy. The intensity of granulysin+ immune infiltration in cervical epithelium did not change significantly between Week 0 and Week 36 in any group (data not shown). However, perforin+ immune infiltrates increased significantly at Week 36 compared to study entry, only in the VGX-3100 treated patients who met primary or secondary endpoints (FIGS. 18A to 18C). This finding correlated directly with the immune signature noted in the peripheral blood.

Taken together, these data confirm that treatment with VGX-3100 drove immune infiltration into cervical lesions and that increased infiltration of lymphocytes with a cytotoxic signature marked specifically by perforin expression was discriminatory in favor of histologic regression of cervical HSIL and clearance of HPV16/18 infection. Expression of perforin but not granulysin was discriminatory, suggesting a possible specificity for the activity of perforin in the context of lesion regression that is not applicable to granulysin.

Materials and Methods

In Situ Hybridization

In situ hybridization was performed using formalin-fixed, paraffin-embedded tissue sections. Biotin-labeled HPV probe solutions (Dako Corporation, Carpinteria, Calif.) were applied to individual sections. These included separate type specific probes for HPV 16 and HPV 18. Detection of hybridized probe was performed by tyramide catalyzed signal amplification utilizing the Dako Genpoint Kit (Dako) per the manufacturer's instructions. Chromogenic detection was performed with DAB/H₂O₂. Controls included tissue sections positive for the HeLa cell line for HPV 18 and the SiHa cell line for HPV 16. Biotin-labeled plasmid probes served as a negative control in each case. Cases with a discrete punctuate reaction product specifically in tumor cell nuclei were interpreted as positive.

Lytic Granule Loading Assay

The lytic granule loading assay was performed as previously described(44) with the exception that CD137 was used in place of HLA-DR and CD38. PBMCs were recovered from cryopreservation overnight in cell culture medium and spun, washed and resuspended the following day. After counting, 1×10⁶ PBMCs were plated into a 96-well plate in R10 medium. For antigen specific responses, cells were stimulated 5 days with a combination of 15-mer peptides overlapping by 8 amino acid residues corresponding to HPV16 E6 and E7 or HPV18 E6 and E7 that had been pooled at a concentration of 2 μg/ml, while an irrelevant peptide was used as a negative control (OVA) and concanavalin A was used as a positive control (Sigma-Aldrich). All peptides were resuspended using DMSO. No costimulatory antibodies or cytokines were added to cell cultures at any point. At the end of the 5 day incubation period, plates were spun to pellet cells and all samples were washed with phosphate buffered saline and subjected to staining for CD3-APCH7, CD4-PerCPCy5.5, CD14-Pacific Blue, CD-16 Pacific Blue, CD137-APC, Granulysin-FITC (BD Biosciences), CD8-BV605 and Granzyme A-AF700 (BioLegend), CD-19 Pacific Blue, granzyme B-PETR (Invitrogen) and perforin-PE (Abcam). Staining for extracellular markers (CD4, CD8, CD137) occurred first, followed by permeablization to stain for the remaining markers. CD3 was stained intracellularly to account for downregulation of the marker following cellular activation. Prepared cells were acquired using an LSR II flow cytometer equipped with BD FACSDiva software (BD Biosciences). Acquired data was analyzed using the FlowJo software version X.0.7 or later (Tree Star).

Immunohistochemistry

Immunohistochemistry was performed as previously described(44). In patients with visible residual disease, an excisional procedure was performed. In patients with no obvious residual disease, a biopsy was obtained at the site of the original lesion. All IHC assays employed a polymer/multimer based secondary detection system. In the case of labvision assays: Vector ImmPRESS HRP appropriate for the species in which the primary antibody was raised was used for detection. For the Roche Ventana assays: Roche UltraView (a Universal kit using anti-mouse & anti-rabbit IgG/HRP conjugate cocktail) was used. For each IHC staining run, irrespective of which platform was used, positive control tissues were included that were treated with the primary antibody and a Buffer Negative where the primary antibody was omitted and acted as a negative control. Samples were initially allocated first for staining CD8 and Foxp3. Remaining tissue was then allocated for staining CD137, CD103, granulysin, PD-L1 and perforin. Differences in number of patients stained for each marker are due to sample availability. Whole slide image capture was performed by Histologix (Biocity Nottingham, UK) at ×20 magnification with a Hamamatsu Nanozoomer 1.0-HT digital slide scanner. Normal and dysplastic epithelium and subjacent stroma morphological regions of interest (ROI) were digitally annotated, where present, onto each section image by the study pathologist. Quantitative image analysis of immunohistochemistry (IHC) staining within the annotated ROI was performed by OracleBio (Biocity Scotland, UK) using Definiens Tissue Studio software.

An analysis algorithm was developed to detect positive cellular staining across each tissue image. Within the algorithm, image colours were initially separated into respective stain components e.g. brown and blue. Cells were defined and generated based on the presence of a blue (haematoxylin) stained nucleus. A threshold level based on identified positive and negative staining in control tissues was then applied to the positive brown colour intensity parameter within each cell, above which a cell was defined as positive. The number or area of positive and negative stained cells was then quantified within specific regions of interest across each tissue image.

Statistics

All analyses are post-hoc. Two-tailed Mann-Whitney U tests are used to compare data be groups and two-tailed Wilcoxon Sign Ranked tests to compare data within a group, as indicated. Statistical analyses were carried out using Prism program 6.0 (GraphPad Software Inc., La Jolla, Calif.) and SPSS Stats 22 with the exact p value and bootstrapping modules (IBM Corp. Released 2013. IBM SPSS Statistics for Windows, Version 22.0. Armonk, N.Y.).

1-21. (canceled)

22. A method of treating high-grade (CIN2/3) cervical dysplasia and HPV-16 and/or HPV-18 infection in a subject who is not responsive to an immunostimulatory composition, said method comprising:
(i) administering a first dose of an immunostimulatory composition to a subject,
(ii) obtaining at least one biological sample from said subject,
(iii) performing assays on the at least one biological sample to detect the presence of an intraepithelial lesion cytology and the presence of HPV-16 and/or HPV-18, wherein a combination of no intraepithelial lesion and no HPV-16 and/or HPV-18 is indicative of responsiveness to the immunostimulatory composition, and
(iv) treating said subject who is not responsive to the immunostimulatory composition with one or more ablative or surgical interventions.

23. The method of claim 22, wherein the biological sample is a peripheral blood sample.

24. The method of claim 22, wherein the biological sample is a cytological sample.

25. The method of claim 24, wherein the cytological sample is a cervix sample.

26. The method of claim 22, wherein the biological sample is obtained 14 weeks after the first dose of the immunostimulatory composition is administered.

27. The method of claim 22, wherein the immunostimulatory composition comprises a DNA plasmid encoding at least one antigen of HPV-16, HPV-18, or both.

28. The method of claim 22, wherein the DNA plasmid encodes an E6 and an E7 antigen of HPV-16, an E6 and an E7 antigen of HPV-18, or both.

29. The method of claim 22, wherein the immunostimulatory composition is VGX-3100.

30. The method of claim 22, wherein the one or more ablative or surgical interventions are selected from the group consisting of cryotherapy, cold-knife conization, or loop electrical excision procedure (LEEP).

31. A method of treating high-grade (CIN2/3) cervical dysplasia and HPV-16 and/or HPV-18 infection in a subject who does not initiate an immune response to an immunostimulatory composition, said method comprising:
(i) administering a first dose of an immunostimulatory composition to a subject,
(ii) obtaining at least one biological sample from said subject,
(iii) performing an assay on the at least one biological sample to determine the presence of CD8+ cells, CD137+ cells, or both, wherein the cells express a detectable level of perforin, granzyme A, granzyme B, or granulysin, or any combination thereof, wherein the presence of said cells indicates an immune response was initiated in response to the immunostimulatory composition, and
(iv) treating said subject who is not responsive to the immunostimulatory composition with one or more ablative or surgical interventions.

32. The method of claim 31, wherein the biological sample is a peripheral blood sample.

33. The method of claim 31, wherein the biological sample is a cytological sample.

34. The method of claim 33, wherein the cytological sample is a cervix sample.

35. The method of claim 31, further comprising testing the sample for the presence of PD-L1.

36. The method of claim 31, wherein the biological sample is obtained 14 weeks after the first dose of the immunostimulatory composition is administered.

37. The method of claim 31, wherein the immunostimulatory composition comprises a DNA plasmid encoding at least one antigen of HPV-16, HPV-18, or both.

38. The method of claim 31, wherein the DNA plasmid encodes an E6 and an E7 antigen of HPV-16, an E6 and an E7 antigen of HPV-18, or both.

39. The method of claim 31, wherein the immunostimulatory composition is VGX-3100.

40. The method of claim 31, wherein the one or more ablative or surgical interventions are selected from the group consisting of cryotherapy, cold-knife conization, or loop electrical excision procedure (LEEP).

41. A method of treating high-grade (CIN2/3) cervical dysplasia and HPV-16 and/or HPV-18 infection in a subject, said method comprising:
(i) administering an immunostimulatory composition to a subject,
(ii) obtaining at least one biological sample from said subject,
(iii) performing assays on the at least one biological sample to detect the presence of an intraepithelial lesion cytology and the presence of HPV-16 and/or HPV-18, wherein a combination of no intraepithelial lesion and no HPV-16 and/or HPV-18 is indicative of responsiveness to the immunostimulatory composition, and
(iv) continuing administration of the immunostimulatory composition to said subject who is responsive to the immunostimulatory composition.
